(19) United States
(12) Patent Application Publication
(10) Pub. No.: US 2024/0065516 A1
(43) Pub. Date: Feb. 29, 2024

(54) UVC LIGHT FOR WASH AND/OR RINSE CYCLES OF DISHWASHERS

(71) Applicant: K.D.W. Company Limited, Hong Kong. SAR. (CN)

(72) Inventors: Kwok Din WONG, Hong Kong (CN); Sai Hin WONG, Hong Kong (CN); Hang Yan WONG, Hong Kong (CN)

(21) Appl. No.: 17/823,538

(22) Filed: Aug. 31, 2022

(51) Int. Cl. A47L 15/42; A47L 15/00; A61L 2/10

(52) U.S. Cl. CPC A47L 15/4242; A47L 15/0005; A47L 15/0007; A47L 15/0015; A61L 2/10; A61L 2202/11; A61L 2202/122; A61L 2202/17

(57) ABSTRACT

A method of washing dishes comprising providing a housing having an interior wash space with dishes positioned therein; spraying a wash liquid onto the dishes positioned within the interior wash space with at least one spray nozzle; spraying a rinse liquid onto the dishes positioned within the interior wash space with at least one spray nozzle after spraying the wash liquid onto the dishes; and emitting ultraviolet (UV) light onto the dishes during the step of spraying the wash liquid onto the dishes, during the step of spraying the rinse liquid onto the dishes, or during both steps of spraying the wash liquid onto the dishes and spraying the rinse liquid onto the dishes.

### FIELD OF THE INVENTION

This disclosure relates generally to the field of dishwashers, and in particular to use of UVC light during a wash and/or rinse cycle.

### BACKGROUND OF THE INVENTION

Commercial establishments for cooking and/or preparing food typically have a commercial dishwasher for washing dirty dishes. The dishwashers spray a detergent and water onto the dirty dishes to clean the dishes. The dishwashers can also spray a rinse aid and water onto the dishes after the dishes have been washed to rinse the dishes and prevent spots on the dishes. Domestic dishwashers are similar, except that domestic dishwashers typically do not add a rinse aid during a rinse cycle. In some domestic dishwashers, a hot air blower is employed to dry the dishes after washing and rinsing.

The prior art includes several methods of sanitizing the dishes after the wash and rinse cycle. CN206007189U, CN105996945A and U.S. Patent Application Publication No. 2009/0110594 all disclose using ultraviolet light during the drying cycle, which comes after the wash cycle and the rinse cycle, to assist in sanitizing the dishes. In these references, the drying and sanitizing occur simultaneously, typically in combination with blown hot air to help dry the dishes while removing humid gas from the dishwasher.

A more efficient dishwasher is desired.

### SUMMARY OF THE INVENTION

The present invention, according to one aspect, is directed to a method of washing dishes comprising providing a housing having an interior wash space with dishes positioned therein; spraying a wash liquid onto the dishes positioned within the interior wash space with at least one spray nozzle; spraying a rinse liquid onto the dishes positioned within the interior wash space with at least one spray nozzle after spraying the wash liquid onto the dishes; and emitting ultraviolet (UV) light onto the dishes during the step of spraying the wash liquid onto the dishes, during the step of spraying the rinse liquid onto the dishes, or during both steps of spraying the wash liquid onto the dishes and spraying the rinse liquid onto the dishes.

Yet another aspect of the present invention is to provide a method of washing dishes comprising providing a housing having an interior wash space with dishes positioned therein; spraying a wash liquid onto the dishes positioned within the interior wash space with at least one spray nozzle; spraying a rinse liquid onto the dishes positioned within the interior wash space with at least one spray nozzle after spraying the wash liquid onto the dishes; fixing an ultraviolet (UV) light housing to a wall of the dishwasher; emitting ultraviolet (UV) light having a wavelength of 100-280 nm through the wall of the dishwasher and onto the dishes during the step of spraying the wash liquid onto the dishes, during the step of spraying the rinse liquid onto the dishes, or during both steps of spraying the wash liquid onto the dishes and spraying the rinse liquid onto the dishes; and diffracting the UV light passing through the wall.

### BRIEF DESCRIPTION OF THE DRAWINGS

One or more embodiments of the present invention are illustrated by way of example and should not be construed as being limited to the specific embodiments depicted in the accompanying drawings, in which like reference numerals indicate similar elements.

FIG. 1 is a front perspective view of a dishwasher according to an embodiment with a door removed for reference.

FIG. 2 is an exploded front perspective view of the dishwasher according to an embodiment with the door removed for reference.

FIG. 3 is an exploded front perspective view of a top portion of the dishwasher.

FIG. 4 is a perspective view of a UV light system of an embodiment.

FIG. 5 is a perspective bottom view of the UV light system of an embodiment.

FIG. 6 is a perspective bottom view of the UV light system of another embodiment.

Certain terminology will be used in the following description for convenience in reference only and will not be limiting. Said terminology will include the words specifically mentioned, derivatives thereof, and words of similar import.

### DETAILED DESCRIPTION

Reference will now be made in detail to implementations and embodiments of various aspects and variations of the invention, examples of which are illustrated in the accompanying drawings. Although at least two variations of the systems, methods and uses are described, other variations of the systems, methods and uses may include aspects of the systems, methods and uses described herein combined in any suitable manner having combinations of all or some of the aspects described.

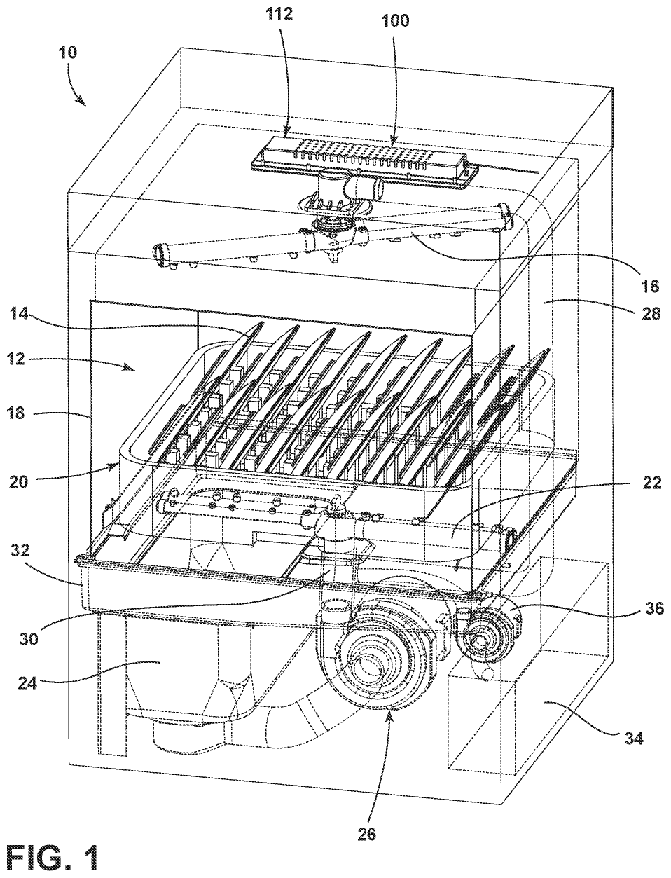

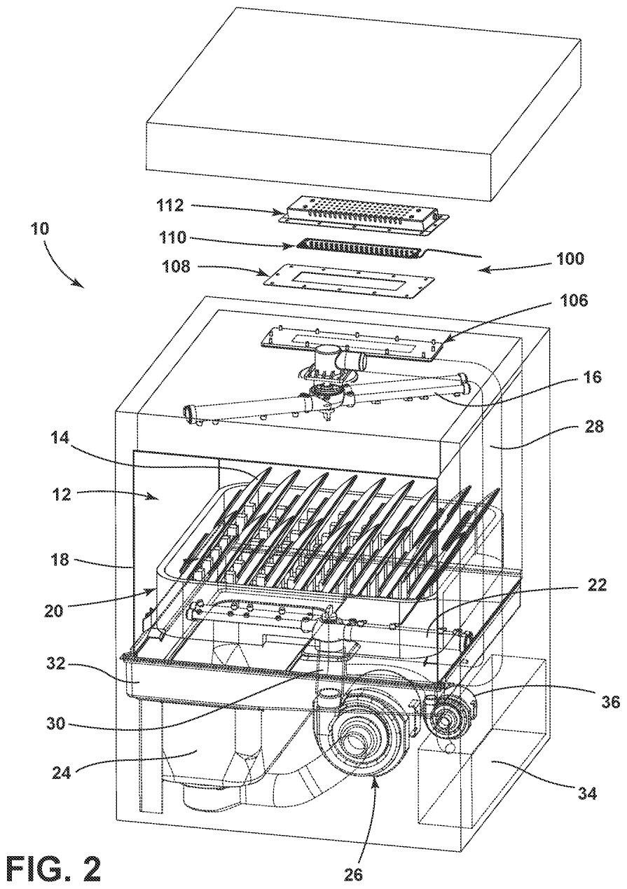

FIGS. 1-2 illustrate an embodiment of a dishwasher or dish washing machine 10. The dishwasher 10 includes an interior wash space 12 for accepting dishes (e.g., glasses, plates 14 (as shown), eating and serving utensils, etc.) for cleaning the dishes. While not shown, the interior wash space 12 can include at least one shelf or ledge as is known to those skilled in the art for directly holding the dishes or holding a tray or rack 20 holding the dishes. The dishwasher 10 includes an opening 18 that is covered by a hinged or sliding door (not shown) to allow access to the interior wash space 12 for placing the dishes into and removing the dishes from the interior wash space 12.

In the illustrated example, the interior wash space 12 of the dish washing machine 10 includes a top rotating spray arm 16 located at a top of the interior wash space 12 for positioning above the dishes (and possibly the tray or rack) along with a bottom rotating spray arm 22 located at a bottom of the interior wash space 12 and below the dishes (and possibly the tray or rack) for spraying water, a washing fluid and/or a rinsing fluid onto the dishes to clean the dishes. Water in a wash tank 24 is pumped by a wash pump 26 to supply the water to the top rotating spray arm 16 and the bottom rotating spray arm 22 to wash and rinse the dishes.

Systems for supplying the water to the interior wash space 12 of the dish washing machine 10 via the top rotating spray arm 16 and the bottom rotating spray arm 22 are well known to those skilled in the art and any such system can be used. For example, the system for supplying water to the interior wash space 12 of the dishwasher 10 via the top rotating spray arm 16 and the bottom rotating spray arm 22 as set forth in U.S. Patent No. 10 905 306 entitled DISH WASHING MACHINE, the entire contents of which are hereby incorporated herein by reference, could be used. Alternatives could also be used. For example, the system could use only one of the top rotating spray arm 16 or the bottom rotating spray arm 22, the arms could be stationary and/or could employ other nozzles (e.g., stationary nozzles extending from interior walls) for spraying water onto the dishes for cleaning the dishes.

In the illustrated embodiment, the dishes are washed by adding water to the wash tank 24. It is also contemplated that the water could be first pre-heated before being supplied to the wash tank 24 using a booster heating tank and/or using heat exchangers. Such booster heating tank and heat exchangers are disclosed in U.S. patent application Ser. No. 17/697,586 entitled DISH WASHING MACHINE WITH HEAT EXCHANGERS, the entire contents of which are hereby incorporated herein by reference. After the water is at the desired temperature in the wash tank 24, the wash cycle begins by sucking the water from the wash tank 24 by a wash pump 26 into a line with a splitter or a pair of lines to bring the water to an upper line 28 that leads to the top rotating spray arm 16 and a lower line 30 that leads to the bottom rotating spray arm 22. The upper line 28 and the lower line 30 can both have a one-way valve on an end thereof to prevent water from returning thereto. The heated water from the upper line 28 and the lower line 30 spray through nozzles in the top rotating spray arm 16 and the bottom rotating spray arm 22, respectively. After leaving the top rotating spray arm 16 and the bottom rotating spray arm 22, the heated water falls to a bottom basin 32 and flows through a bottom hole therein back into the wash tank 24. A wash detergent can be injected into the wash tank 24 to help with the cleaning of the dishes and the water in the wash tank is heated (e.g., to about 65° C. or higher). It is contemplated that wash detergent could be added to the wash tank 24 periodically or continually during the wash process. The wash cycle happens a desired number of times to wash the dishes.

During the wash cycle, the wash water from the upper line 22 and the lower line 24 spray through wash nozzles in the top rotating spray arm 16 and the bottom rotating spray arm 22, respectively. The force of the wash water leaving the wash nozzles forces the top rotating spray arm 16 (spraying downward) and the bottom rotating spray arm 22 (spraying upward) to rotate as is well known to those skilled in the art. Eventually, the wash water falls to the bottom basin 32 and flows through the bottom hole therein into the wash tank 24 to continue the wash cycle. After the illustrated washing cycle is complete, the wash water is removed from the dish washer 10 through a drain pipe (not shown) by force of a discharge pump and/or gravity.

In the illustrated example, after the dishes in the dish washing machine 10 have been washed, the dishes are rinsed. During the rinsing cycle, water is once again supplied to the wash tank 24. The water for the rinse cycle can be heated in the wash tank 24 and/or pre-heated before being supplied to the wash tank 24 as outlined above. A rinse aid is injected into the wash tank 24 to help with the rinsing of the dishes. As shown in FIGS. 1-2, a rinse tank 34 holds rinse aid, a rinse pump 36 sucks the rinse aid from the rinse tank 34 and supplies the rinse aid to the wash tank 24 (which at this point does not include any cleaning detergent in the water). The rinse aid can be a surfactant that lowers a surface tension of water, so that the water more easily slides off dishes in the dishwasher 10.

Aspects of the present embodiment include a method of using ultraviolet (UV) light during the wash and/or rinse cycle. Therefore, UV light is emitted from a UV light system 100 at the same time wash water and/or the rinse water (with or without rinse aid) is passing through the system during the rinse cycle as outlined above. The UV light emitted from the UV light system 100 is preferably ultraviolet C (UVC), which has a wavelength of 100-280 nm. The UVC light disinfects the dishes 14 from microbial contaminants.

In the illustrated embodiment, the UV light system 100 emits UV light (e.g., UVC light) into the interior wash space 12 to disinfect the dishes 14 during the wash cycle and/or the rinse cycle. The UV light system 100 is illustrated in FIGS. 1-3 as being located at a top of the dishwasher 10 to emit the UV light through a top wall 204 (e.g., made of metal) of the dishwasher 10 into the interior wash space 12. While only a single UV light system 100 is shown, it is contemplated that any number of UV light systems 100 could be employed. Moreover, while the UV light system 100 is shown as emitting light through the top wall 204 of the dishwasher 10, the UV light system 100 could be located to emit light through any wall of the dishwasher 10 and into the interior wash space 12, including the side walls, the rear wall, the interior of the door and/or the wall surrounding the door.

The illustrated UV light system 100 is configured to be sealed from the liquid within the dishwasher 10 during the wash and rinse cycles, but emit the UV light into the dishwasher 10. The UV light system 100 includes an exterior seal plate 104, a lens 106, an interior seal plate 108, a circuit board 110 and a cover 112. The UV light system 100 rests on a wall of the dishwasher 10 and liquid is prevented from reaching the circuit board 110 as outlined below.

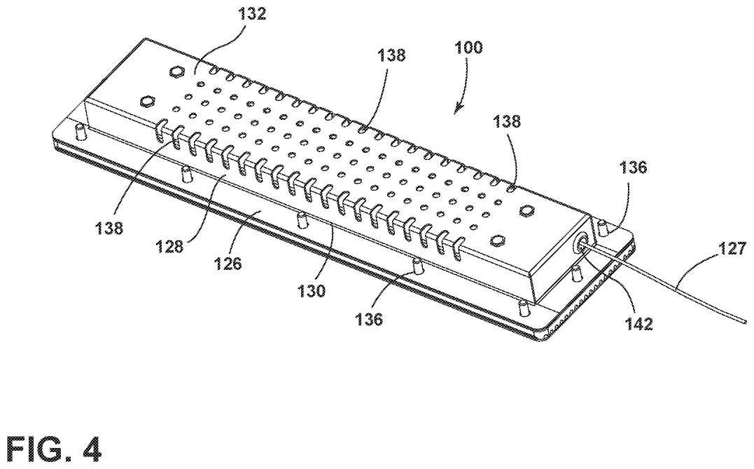

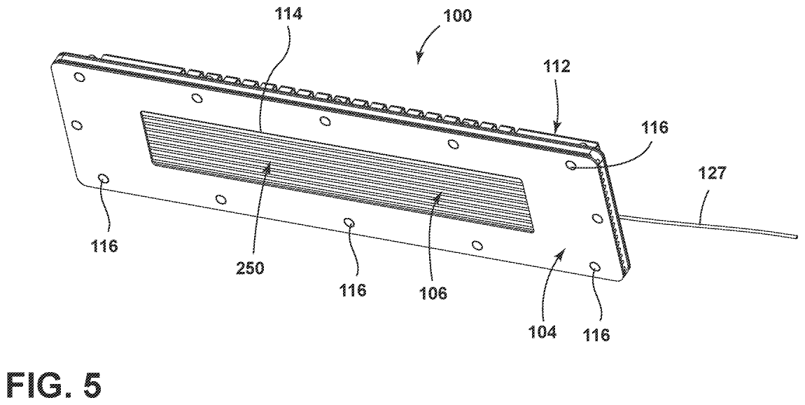

In the illustrated embodiment, the lens 106 is sandwiched between the exterior seal plate 104 and the interior seal plate 108 as shown in FIGS. 1, 4 and 5. The exterior seal plate 104 and the interior seal plate 108 are substantially identical, but can have different thicknesses and be made of different material if desired. The exterior seal plate 104 is substantially rectangular and includes a central opening 114 (e.g., rectangular). A plurality of holes 116 surround the central opening 114. Likewise, the interior seal plate 108 is substantially rectangular and includes a central opening 118 (e.g., rectangular). A plurality of holes 120 surround the central opening 118. The lens 106 has a substantially rectangular periphery and includes a plurality of apertures 122 located adjacent the periphery thereof. When the lens 106 is sandwiched between the exterior seal plate 104 and the interior seal plate 108, the holes 116 and the holes 120 of the exterior seal plate 104 and the interior seal plate 108, respectively, line up with each other and with the apertures 122 in the lens 106. The exterior seal plate 104 and the interior seal plate 108 are configured to be substantially resistant to UV light.

The illustrated circuit board 110 has UV light emitters 124 (e.g., LEDs) thereon for emitting the UV light. The circuit board 110 is substantially rectangular and is configured to be positioned within the central opening 118 of the interior seal plate 108. It is contemplated that the circuit board 110 could be replaced with other elements for emitting UV light, such as supports for an incandescent bulb or a fluorescent tube. A power cord 127 is connected to the circuit board 110 to supply power to the UV light emitters 124 as is well known to those skilled in the art. The power cord 127 is connected to a control system to the dishwasher 10 as is well known to those skilled in the art, with the control system powering the UV light emitters 124 at the desired time during the process as described herein. The cover 112 covers the circuit board 110. The cover 112 includes a flat rectangular lip 126, a rectangular wall 128 extending upward from an interior edge 130 of the flat rectangular lip 126, and a top plate 132 connected to the edge of the rectangular wall 128 opposite the flat rectangular lip 126. The flat rectangular lip 126 includes a plurality of holes 134 therethrough. The holes 134 align with the holes 116 and the holes 120 of the exterior seal plate 104 and the interior seal plate 108, respectively, and the apertures 122 in the lens 106 to form throughhole openings 136. The cover 112 includes a plurality of vents 138 in the top plate 132, the rectangular wall 128, and/or at an intersection of the top plate 132 and the rectangular wall 128 to allow heat to vent from within the cover 112. The rectangular wall 128 has an outlet opening 140 for allowing the power cord 127 to pass therethrough. The power cord 127 can have a circular seal 142 for sealing the outlet opening 140 and for maintaining the power cord 127 in position relative to the cover 112.

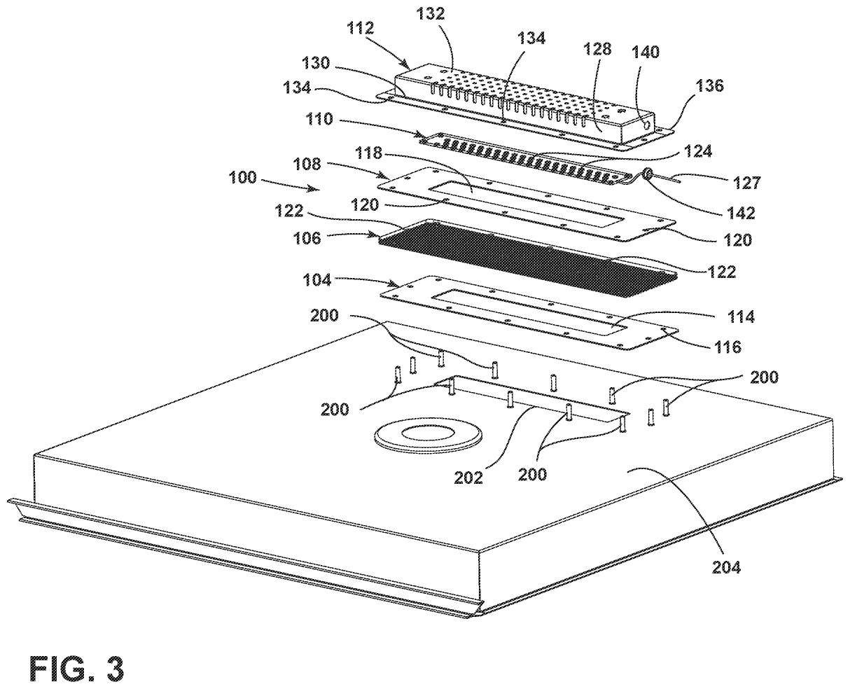

The UV light system 100 can be connected to the dishwasher 10 in any manner. In the illustrated embodiment, the UV light system 100 is connected to the dishwasher 10 by extending fasteners 200 surrounding an opening 202 in the top wall 204 of the dishwasher 10 (see FIG. 3) through the UV light system 100 throughhole openings 136. Nuts or other elements can be inserted over the ends of the fasteners 200 to lock the UV light system 100 in position.

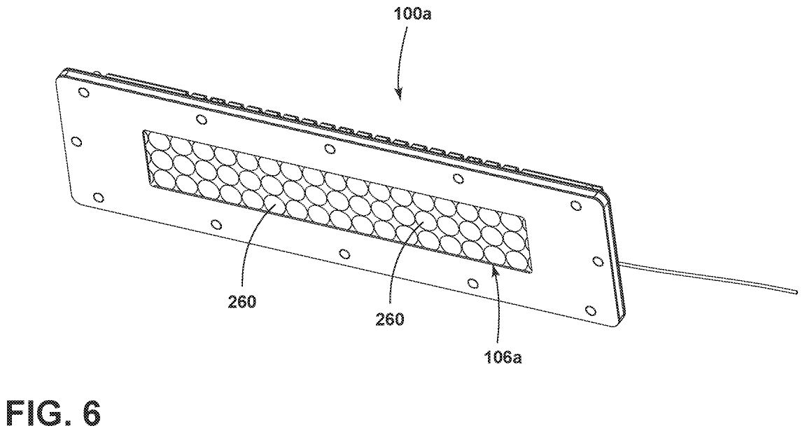

In the illustrated example, the lens 106 is configured to allow the UV light to pass therethrough. For example, the lens 106 can be made of glass (e.g., quartz glass). The lens 106 allows the UV light to pass therethrough and prevents liquid from leaking out of the dishwasher 10. Furthermore, the lens 106 can be textured to disperse the UV light passing therethrough. For example, the lens 106 can have surfaces that disperse and/or refract light passing through the lens. The lens 106 for the UV light system 100 in FIGS. 1-5 has a diffracting surface 250. The UV light system 100a as shown in FIG. 6 is identical to the UV light system 100 of FIGS. 1-5, except that the lens 106a has interior cells 260 that diffract the UV light passing therethrough.

In the dishwasher 10 as disclosed herein, the UV light system 100, 100a emits UV light (e.g., UVC light) into the interior wash space 12 of dishwasher 10 during the wash cycle, during the rinse cycle, or during both the wash cycle and the rinse cycle. The UV light passing through the lens 106, 106a is refracted and/or dispersed to emit the light in many directions to sufficiently contact the dishes 14.

Although particular preferred embodiments of the invention have been disclosed in detail for illustrative purposes, it will be recognized that variations or modifications of the disclosed apparatus, including the rearrangement of parts, lie within the scope of the present invention. For example, it is contemplated that only a single rotating spray arm (upper or lower) could be used.

What is claimed is:

1. A method of washing dishes comprising:
providing a housing having an interior wash space with dishes positioned therein;
spraying a wash liquid onto the dishes positioned within the interior wash space with at least one spray nozzle;
spraying a rinse liquid onto the dishes positioned within the interior wash space with at least one spray nozzle after spraying the wash liquid onto the dishes; and
emitting ultraviolet (UV) light onto the dishes during the step of spraying the wash liquid onto the dishes, during the step of spraying the rinse liquid onto the dishes, or during both steps of spraying the wash liquid onto the dishes and spraying the rinse liquid onto the dishes.

2. The method of claim 1, wherein:
the step of emitting ultraviolet (UV) light onto the dishes only occurs during the step of spraying the wash liquid onto the dishes.

3. The method of claim 1, wherein:
the step of emitting ultraviolet (UV) light onto the dishes only occurs during the step of spraying the rinse liquid onto the dishes.

4. The method of claim 1, wherein:
the step of emitting ultraviolet (UV) light onto the dishes occurs during both steps of spraying the wash liquid onto the dishes and spraying the rinse liquid onto the dishes.

5. The method of claim 1, wherein:
the wash liquid includes a wash detergent.

6. The method of claim 1, wherein:
the rinse liquid includes a rinse aid.

7. The method of claim 1, wherein:
the UV light has a wavelength of 100-280 nm.

8. The method of claim 1, further including:
fixing a UV light housing to a wall of a dishwasher, wherein the UV light is emitting from the UV light housing through the wall of the dishwasher.

9. The method of claim 8, wherein:
the UV light housing includes UV light emitters and a window, the window forming a portion of the wall of the dishwasher separating the UV light emitters from the interior wash space.

10. The method of claim 9, further including:
diffracting the UV light passing through the window.

11. The method of claim 8, wherein:
the UV light housing includes an outer seal, a lens, an inner seal, UV light emitters and a vented cover, with the outer seal and the lens preventing liquid from entering an interior of the UV light housing.

12. A method of washing dishes comprising:
providing a housing having an interior wash space with dishes positioned therein;
spraying a wash liquid onto the dishes positioned within the interior wash space with at least one spray nozzle;
spraying a rinse liquid onto the dishes positioned within the interior wash space with at least one spray nozzle after spraying the wash liquid onto the dishes;
fixing an ultraviolet (UV) light housing to a wall of a dishwasher;
emitting ultraviolet (UV) light having a wavelength of 100-280 nm through the wall of the dishwasher and onto the dishes during the step of spraying the wash liquid onto the dishes, during the step of spraying the rinse liquid onto the dishes, or during both steps of spraying the wash liquid onto the dishes and spraying the rinse liquid onto the dishes; and
diffracting the UV light passing through the wall.

13. The method of claim 12, wherein:
the step of emitting ultraviolet (UV) light onto the dishes only occurs during the step of spraying the wash liquid onto the dishes.

14. The method of claim 12, wherein:
the step of emitting ultraviolet (UV) light onto the dishes only occurs during the step of spraying the rinse liquid onto the dishes.

15. The method of claim 12, wherein:
the step of emitting ultraviolet (UV) light onto the dishes occurs during both steps of spraying the wash liquid onto the dishes and spraying the rinse liquid onto the dishes.

16. The method of claim 12, wherein:
the wash liquid includes a wash detergent.

17. The method of claim 12, wherein:
the rinse liquid includes a rinse aid.

18. The method of claim 12, wherein:
the UV light housing includes UV light emitters and a window, the window forming a portion of the wall of the dishwasher separating the UV light emitters from the interior wash space.

19. The method of claim 12, wherein:
the UV light housing includes an outer seal, a lens, an inner seal, UV light emitters and a vented cover, with the outer seal and the lens preventing liquid from entering an interior of the UV light housing.
